(19) United States
(12) Patent Application Publication
(10) Pub. No.: US 2018/0000745 A1
(43) Pub. Date: Jan. 4, 2018

(54) CRUCIFERIN COATED NANOPARTICLES

(71) Applicants: Jianping WU, Edmonton (CA); Ali AKBARI, Edmonton (CA)

(72) Inventors: Jianping WU, Edmonton (CA); Ali AKBARI, Edmonton (CA)

(21) Appl. No.: 15/198,738

(22) Filed: Jun. 30, 2016

(51) Int. Cl. A61K 9/51; A23L 33/10; A23L 33/17; A61K 35/741

(52) U.S. Cl. CPC A61K 9/5169; A61K 35/741; A61K 9/5192; A23L 33/10; A61K 9/5161; A23L 33/17; A23V 2002/00

(57) ABSTRACT

A method for preparation of cruciferin-coated chitosan particles as a carrier for encapsulation and delivery of heat, pH and/or protease-degradable hydrophilic or hydrophobic bioactive compounds.

### FIELD OF THE INVENTION

The present invention relates to chitosan nanoparticles coated with an edible canola protein, and a process for coating and protection of chitosan particles with an edible canola protein. The coated chitosan particles can be used for encapsulation and delivery of bioactive material and drugs.

### BACKGROUND OF THE INVENTION

Functional foods and nutraceuticals have potential to improve human health and wellness; however the low bioavailability of many bioactive food compounds makes this potential health benefit difficult to achieve. Encapsulation of bioactive compounds is recognized as an effective approach to improve bioavailability by providing protection against food processing and digestion in the gastrointestinal (GI) tract, and improving their solubility and/or permeability in the GI tract. Chitosan has been explored as a wall material for preparing particles due to its unique mucoadhesive and permeation enhancing properties. However, chitosan is degraded at low pH. To overcome this hurdle, chitosan particles are often coated with synthetic co-polymers such as Eudragit™, which is commercially available in many different forms. However, in food uses, natural polymers such as food proteins, are more desirable compared to the synthetic ones.

### SUMMARY OF THE INVENTION

In one aspect, the invention comprises a nanoparticle comprising cruciferin, chitosan and a bioactive material. The bioactive material may be a water soluble or insoluble compound or a probiotic, and may be incorporated into the nanoparticle during formation of the nanoparticle.

In another aspect, the invention may comprise a method of forming a nanoparticle for delivery of a bioactive material, comprising the steps of:
(a) combining a solution of cruciferin and a solution of chitosan in a weight ratio greater than 1:1 and less than about 5:1, and at a pH greater than 4.5 and less than 7.5; and
(b) collecting the resulting nanoparticles.

Preferably, the pH of the combined solution is adjusted to between about 5.5 to about 6.5, and the ratio of cruciferin to chitosan is in the range of about 3:2 to about 5:2 by weight.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1 shows TEM images of cruciferin solution (5 mg/mL) (a); chitosan solution (2 mg/mL) (b); cruciferin-coated chitosan particles suspension (c). Samples were diluted 10 times before observation.

FIG. 2. Intrinsic fluorescence intensity of cruciferin and cruciferin-coated chitosan particles.

FIG. 3. The effect of DTT (a), urea (b) and NaCl (c) on the turbidity of the nanoparticles dispersions.

FIG. 4. DSC thermograms of cruciferin, chitosan and cruciferin-coated chitosan particles: melting points (a) and water evaporation temperatures (b).

FIG. 5. FTIR spectra (a) and deconvoluted FTIR spectra (b) of cruciferin, chitosan and cruciferin-coated chitosan particles.

FIG. 6. Effect of cruciferin-coated chitosan nanoparticles on Caco-2 cells survival after 24 h incubation at 37° C. (compared to control).

FIG. 7. Confocal microscopic images of Caco-2 cells after 6 h incubation with coumarin 6-loaded cruciferin-coated chitosan nanoparticles (green) (A) and free coumarin-6 (B). The cell membrane and nucleus were stained using Alexa 594 and DAPI, respectively.

FIG. 8. Release profiles of brilliant blue (a) and β-carotene (b) from cruciferin-coated chitosan particles in simulated gastric fluid (SGF) followed by simulated intestinal fluid (SIF) in the presence and absence of pepsin and pancreatin, respectively.

FIG. 9. DSC thermograms of β-carotene, cruciferin-coated chitosan particles and β-carotene-loaded particles: melting points (a) and water evaporation temperatures (b).

FIG. 10. FTIR spectra of cruciferin-coated chitosan particles, β-carotene and β-carotene-loaded particles.

FIG. 11. Effect of encapsulation of different β-carotene concentrations on intrinsic fluorescence intensity of cruciferin-coated chitosan particles

### DETAILED DESCRIPTION OF PREFERRED EMBODIMENTS

In one embodiment, the invention comprises the use of an edible canola protein as a functional coating to produce a nanoparticle delivery system. Most food proteins form heat-induced aggregations at temperatures above about 70° C., in which different compounds can be entrapped and slowly be released. Since heat-set hydrogels may not be suitable for encapsulation of heat-sensitive compounds, an alternative method, cold gelation, in which proteins form a gel using multivalent ions or in combination with other polymers, may be applied. In one specific embodiment, cruciferin is used to coat chitosan particles, resulting in particles with improved stability at low pH.

As used herein, a nanoparticle is an agglomeration comprising cruciferin and chitosan molecules, having at least one dimension less than about 1000 nm, and preferably less than about 500 nm. Preferably, the nanoparticle is roughly spherical, with cruciferin forming a complete or partial shell around a core of chitosan. A bioactive material may be incorporated into the nanoparticle, and may be associated with the chitosan, wholly or partially encapsulated by cruciferin. A bioactive material may be any compound or combination of compounds which is intended to be consumed by a patient or subject and which may have a therapeutic or biological effect. A bioactive material may include probiotic bacteria or viruses. The bioactive compound may be a pharmaceutical or nutraceutical compound.

Cruciferin is the major canola protein (11S or 12S globulin) accounting for ˜65% of total canola proteins. The meal remaining after oil extraction contains 35-40% proteins, and canola proteins are considered potential food proteins as they contain a well-balanced amino acid composition, high contents of lysine (6.0%) and sulfur-containing amino acids (3 to 4%), and a high protein efficiency ratio. Furthermore, canola proteins have been shown to exhibit emulsifying and gelling properties, and may be broken down into bioactive peptides. Cruciferin, with an isoelectric point (IEP) of around 7.2 and a molecular weight of about 300 kDa, is composed of six subunits and is resistant to gastric digestion. As used herein, cruciferin may comprise all isoforms and variants thereof, including recombinant variants, which form nanoparticles with chitosan in a cold-gelation type method.

Chitosan is a water-soluble, linear polysaccharide derived from chitin of fungi cell walls or exoskeleton of crabs and shrimp, comprising a copolymer of acetylated and deacetylated glucosamine. Chitosan may be produced by the deacetylation of chitin, where the degree of deacetylation may vary from about 50% to about 100%. Chitosan may have an average molecular weight (number or weight average) in the range of about 3800 to about 20,000 Daltons. As used herein, chitosan includes any of its derivatives or variants which form nanoparticles with cruciferin in a cold-gelation type method.

In one preferred embodiment, cruciferin-coated chitosan nanoparticles are prepared. Such particles may have an average size of 50 to 500 nm, and preferably in the range of about 100 to 200 nm. The nanoparticles may be prepared in solution at a pH of between about 4.5 to about 6.5, preferably about 5.5, and a cruciferin:chitosan ratio of between about 3:2 to about 5:2 (by weight). The cruciferin shell surrounds the chitosan core in the particles. The particles may encapsulate a bioactive material. The cruciferin-coated chitosan nanoparticles particles may protect the bioactive material against pH and enzymatic degradation in the stomach and intestine, thus, they may serve as carriers for compounds sensitive to stomach and intestinal conditions.

In addition to the controlled release of the compounds in the intestine, the nanoparticles may also be absorbed directly by cells, thereby improving bioavailability due to the mucoadhesive and paracellular permeation enhancing properties of chitosan. Moreover, in another embodiment, the nanoparticles might also act as colon targeting delivery systems since chitosan is degraded in the colon and release the encapsulated bioactive material in the colon. Since cruciferin is a GRAS natural polymer, bioactive-loaded particles might be used for food fortification; this is the advantage of the particles compared to synthetic polymers-coated chitosan particles.

Exemplary embodiments of the present invention are described in the following Examples, which are set forth to aid in the understanding of the invention, and should not be construed to limit in any way the scope of the invention as defined in the claims which follow thereafter. As will be apparent to those skilled in the art, various modifications, adaptations and variations of the specific disclosure herein can be made without departing from the scope of the invention claimed herein.

### Example 1—Materials

Commercial canola meal, obtained from Richardson Oilseed Company (Lethbridge, AB, Canada) was ground, passed through a 35-mesh screen and stored at −20° C. for further use. Caco-2 cells (HTB37) were obtained at passage 19 from the American type culture collection (Manassas, Va.). Dulbecco's modified eagle medium (DMEM), 0.25% (w/v) trypsin-0.53 mM EDTA, 4-(2-hydroxyethyl)-1-piperazineethanesulfonic acid (HEPES), fetal bovine serum, 1% nonessential amino acids, and 1% antibiotics were all procured from Gibco Invitrogen (Burlington, ON, Canada). (3-(4,5-dimethylthiazol-2-yl)-2,5-diphenyltetrazolium bromide) (MTT), dimethyl sulfoxide (DMSO), coomassie brilliant blue G-250, β-carotene, chitosan (molecular weight of 140-220 kDa and degree of acetylation ≦40%), urea, sodium dodecyl sulfate (SDS), dithiothreitol (DTT), pepsin, pancreatin, Coumarin 6 and 4′,6-diamidino-2-phenylindole (DAPI) were obtained from Sigma (Oakville, ON, Canada).

### Example 2—Canola Protein Extraction

Cruciferin was extracted using the method previously developed and described (Akbari et al. 2015) A slurry of canola meal:water (1:10, W:V) was acidified to pH 4, stirred for 2 h and centrifuged at 10,000 rpm for 20 min at 4° C. The resultant pellet was mixed with 10× volume of water, and the pH was adjusted to 12 while stirring at room temperature for 1 h. After centrifugation, the supernatant was adjusted to pH 4 to precipitate proteins. The collected precipitate was washed twice with acidified water (pH 4), centrifuged and freeze-dried as cruciferin isolate.

### Example 3—Preparation of Particles

To prepare the particles, cruciferin and chitosan were solubilized at different concentrations at alkaline (pH 12) and acidic (pH 3) conditions, respectively, and then the cruciferin solution was dropwise added to the chitosan solution to achieve a pH ˜4.5. The pH of the mixture was adjusted to different pHs and the formed particles suspensions were studied. 18 mL of cruciferin solution in water at different protein concentrations (2, 3, 5, 7.5, and 10 mg/mL) and pH 12 was dropwise added to 18 mL chitosan solution (2 mg/mL in 0.1 M acetic acid) containing 0.04% w/v sodium azide while stirring. The pH of the suspensions was adjusted to 3.5, 4.5, 5.5, 6.5, and 7.5, and the suspensions were stirred for 1 h. The stability and turbidity of the particle suspensions were used to assess conditions for particles formation. The stability of the particles was studied for a period of 3 weeks stored at 4° C. The turbidity was measured at 600 nm using a UV/VIS spectrophotometer (V-530 Jasco, Japan).

Since the formation of particles would increase the turbidity of the suspensions, an indicator of 50% increase in the turbidity compared to initial protein solution was used to determine an appropriate particles formation. Our results showed that the mixtures did not form particles at pHs 3.5 and 4.5 (low turbidity) likely due to high repulsive forces between positively-charged cruciferin and chitosan, formed at pHs 5.5 and 6.5 at certain ratios of cruciferin:chitosan, and precipitated at pH 7.5. At pHs 5.5 and 6.5, in general, the appearance of the particles suspensions changed from clear to opaque at increasing cruciferin concentrations. Whereas the mixtures did not form highly-turbid suspension at the ratio of 2:2 (by weight) and the particles prepared at the ratios of 7.5:2 and 10:2 were not stable and precipitated; the particles prepared at the ratios of 3:2 and 5:2 were stable and therefore used for further studies.

### Example 4—Size, Surface Charge and Morphology of Particles

Size of the particles was determined by dynamic light scattering using Malvern Nanosizer ZS (Malvern, Worcestershire, UK). Zeta potential of the particles was also measured by laser doppler velocimetry using the Nanosizer. Prior to measurement, samples were diluted in 10 mM phosphate buffers (the same pH of particle suspensions) to obtain a slight opalescent dispersion to prevent multiple scattering effects. Morphology of the prepared particles was studied using a transmission electron microscopy (TEM, Philips Morgagni 268, FEI Company, The Netherlands) at 80 kV. The particles were freshly prepared at the cruciferin:chitosan ratio of 5:2 and pH 5.5, diluted 10 times, put on Formvar-covered copper grids, negatively stained with 2% phosphotungstic acid for 20 S, and then air dried.

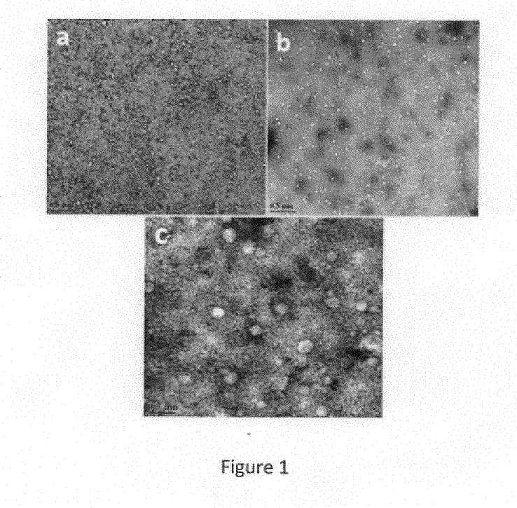

The zeta potential and size of chitosan and cruciferin before forming the particles were ˜+43 mV and 25 nm, and −41 mV and 105 nm at pH 3 and 12, respectively. The surface charge of cruciferin-coated chitosan nanoparticles formed at pH 5.5 with 165 nm size was ˜+18 mV. In general, the particle size increased at increasing pHs from 5.5 to 6.5, likely due to decreased protonation of chitosan (pKa˜6.4) and cruciferin (IEP ˜7.2) (Table 1), which would decrease repulsion forces between chitosan and cruciferin, leading to particle aggregation and even precipitation at increasing cruciferin and chitosan ratio above 5:2 (data not shown). The zeta potential of particles was not affected. Similar trend was reported for chitosan/tripolyphosphate and chitosan/hydroxypropyl methylcellulose phthalate by Makhlof et al, (2011).³ Polydispersity index (PDI) of the particles sizes was ˜0.35, revealing satisfactory particles uniformity. Therefore, the particles prepared at the cruciferin:chitosan ratios of 3:2 and 5:2 at pH 5.5 were appropriate, and the ratio of 5:2 was chosen to prepare particles for further studies. Morphology of cruciferin and chitosan solutions and the particles were examined using TEM, No specific structure was observed in the cruciferin and chitosan solution (FIGS. 1a and 1b) while spherical shapes were formed at 150-200 nm diameter (FIG. 1c).

TABLE 1
Effects of cruciferin:chitosan ratio and pH on increased turbidity, size,
and zeta potential of prepared particles. Values with different lowercase
letters in the same column are significantly (p < 0.05) different.

| cruciferin: | | Increased | Size | Zeta potential |
|---|---|---|---|---|
| chitosan ratio | pH | turbidity (%) | (nm) | (mV) |
| 3:2 | 5.5 | 190 ± 4 ^{c} | 157 ± 8 ^{b} | +20 ± 1.6 ^{a} |
| | 6.5 | 281 ± 1 ^{a} | 175 ± 10 ^{a} | +15.1 ± 2.4 ^{a} |
| 5:2 | 5.5 | 204 ± 1 ^{b} | 165 ± 3 ^{ab} | +18.2 ± 1.3 ^{a} |
| | 6.5 | Precipitated particles | — | — |

### Example 5—Surface Hydrophobicity

The surface hydrophobicity of cruciferin and the particles was determined using ANS fluorescent probe. Briefly, the samples were diluted to ten concentrations ranging from 0.0025 to 0.1 mg/mL using 0.1 M citrate buffer at pH 5. Then, 20 μL of 8 mM ANS solution prepared in the same buffer was added to 4 mL of the diluted samples and the fluorescence intensity was measured at emission and excitation wavelengths of 470 nm and 390 nm, respectively, at a slit width of 5 nm using a 1-cm path length quartz cell on Shimadzu RF-5301PC spectrofluorophotometer (Kyoto, Japan). The buffer containing ANS and diluted samples (in the absence of ANS), as blanks, was subtracted from the measured fluorescence values. The slope of the linear plot of net fluorescence values versus protein concentrations was used as an index of the protein surface hydrophobicity.

The surface hydrophobicity (S₀) of cruciferin was not affected before and after particle formation (8613±3 and 8421±34, respectively), which indicated that the particle formation process didn't affect protein folding/unfolding and/or the availability of hydrophobic groups on the protein surface, Unlike Boeris et al. (2011) who reported chitosan decreased the S₀ of the pepsin/chitosan complex due to blocking the surface hydrophobic groups; the unchanged S₀ during particle formation suggested that either S₀ of cruciferin in the coating layer was not affected by chitosan, or the coating layer was just composed of cruciferin.

### Example 6—Intrinsic Fluorescence Study

The intrinsic fluorescence emissions of cruciferin and the particles were measured using a Shimadzu RF-5301PC spectrofluorophotometer (Kyoto, Japan) equipped with a 1-cm path length quartz cell. The protein concentration and pH of the samples were adjusted to 0.005 mg/mL and pH 5.5 using 10 mM citrate buffer. The fluorescence measurements were performed using the excitation wavelength of 295 nm and the emission wavelengths of 300 to 650 nm, both at a slit width of 5 nm. The interactions between encapsulated β-carotene and the particles were also studied using the intrinsic fluorescence property. In brief, different concentrations of β-carotene, soluble in ethanol, were encapsulated in the particles as will be explained in 2.7.2 section. The intrinsic fluorescence of the β-carotene-loaded particles, at final β-carotene concentrations of 0.002-0.06 mg/mL, was also measured using 295 nm excitation wavelength. The emission spectra were recorded from of 300 to 650 nm wavelength.

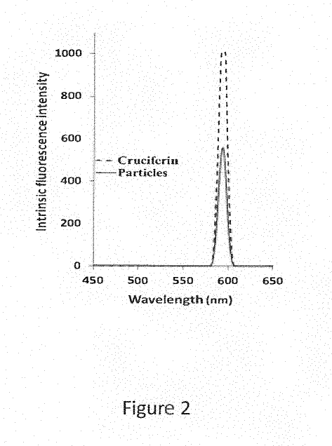

The intrinsic fluorescence of a protein strongly depends on the conformation that the protein adopts in response to bulk solution conditions. Intrinsic fluorescence property of a protein is due to the presence of aromatic amino acids such as tryptophan and tyrosine. The intensity of intrinsic fluorescence property of the particles at 594 nm was lower than that of cruciferin (FIG. 2). Since no shift occurred in the emission λₘₐₓ of cruciferin before and after the complex formation, suggesting that the polarity of the environment surrounding tryptophan residues didn't change and therefore folding/unfolding process didn't happen in the particles formation process, which is in agreement with surface hydrophobicity results. The increased suspension turbidity resulting from the particles formation might be one reason for the decreased emission intensity of the complex since the turbidity could reduce the exciting and/or emitted radiations. However, the effect of the turbidity on emission intensity would be negligible due to the use of extensively diluted samples (×100 times). Binding-induced quenching effect might be responsible for decreased intrinsic emission fluorescence of tryptophan during particle formation. Therefore, the interaction between chitosan and cruciferin might quench the fluorescence property of cruciferin in the particles.

### Example 7—Driving Forces Involving the Particles Formation

The importance of hydrophobic, electrostatic, hydrogen and disulfide interactions in forming the particles were evaluated using SDS, NaCl, urea and DTT, respectively. The particles suspension was mixed with the same volume of each dissociating agent at different concentrations. The mixtures were adjusted to pH 5.5 and vortexed 10 s and after 6 h, their turbidity were measured at 600 nm using the UV/VIS spectrophotometer. The turbidity of the mixtures was compared to that of initial particles suspension. Decrease in the turbidity of the mixtures was considered as an indicator of the degree of particles dissociation.

As described above, adding alkaline cruciferin solution (negatively-charged) into acidic chitosan solution (positively-charged) led to a pH value of ˜4.5, which was lower than IEP of cruciferin (˜7.2) and pKa of chitosan (˜6.4); therefore, both cruciferin and chitosan polymers were positively-charged which would prevent the particle formation due to the presence of repulsive forces. At increasing pHs to 5.5-6, the decreased protonation of the polymers also decreased their repulsive forces, enabling them to come closer to form different interactions.

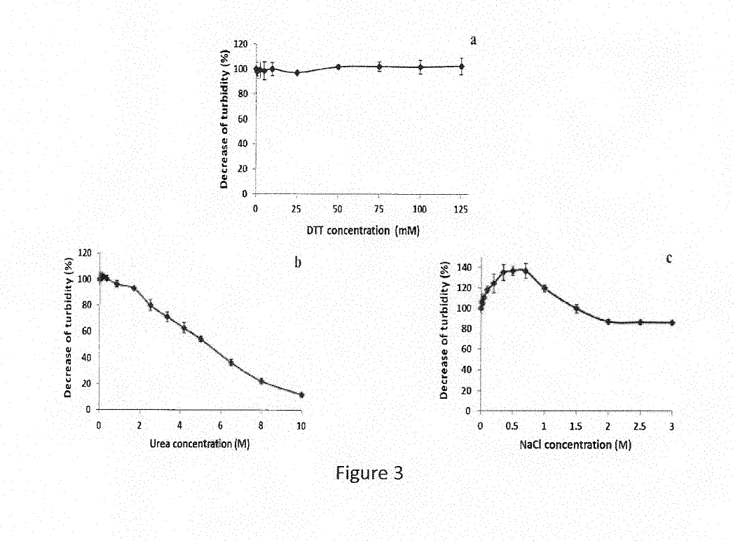

The formation of particles could be affected by a number of interactions such as electrostatic, disulfide, hydrophobic and hydrogen bonding. To understand the responsible force for the particle formation, effects of dissociating agents, namely SDS, DTT, urea and NaCl, on the suspensions turbidity were determined. The suspension turbidity was decreased by 89 and 14% in the presence of urea and NaCl, respectively, but was not affected by DTT (FIG. 3); SDS precipitated the particles due to the attractive interaction with the positively charged particles. The results showed that although electrostatic interaction could influence the particles formation due to the presence of amine and carboxyl groups in chitosan and cruciferin, hydrogen bonding was the major driving force for the particle preparation. Qin et al. (2011) and Xu and Du (2003) also reported that hydrogen bonding was the main force for chitosan particles formation. As it was expected, disulfide bond and hydrophobic interactions were not important in the particles formation due to the absence of sulfhydryl and hydrophobic groups in chitosan. However, it was interesting to note that protein-protein interactions (i.e. hydrophobic forces) do not appear to play critical roles in the particles preparation.

### Example 8—Thermal Property Study

Freeze-dried samples were dried in a vacuum desiccator using phosphorous pentoxide for 48 h. The dried samples (˜5 mg) were weighed in hermetically sealed aluminium pans, sealed and loaded into a differential scanning calorimeter (Q2000-DSC, TA Instruments, New Castle, Del., USA). The samples were heated at a scanning rate of 10° C./min from 15 to 300° C. under inert nitrogen atmosphere. An empty aluminium pan was used as a reference.

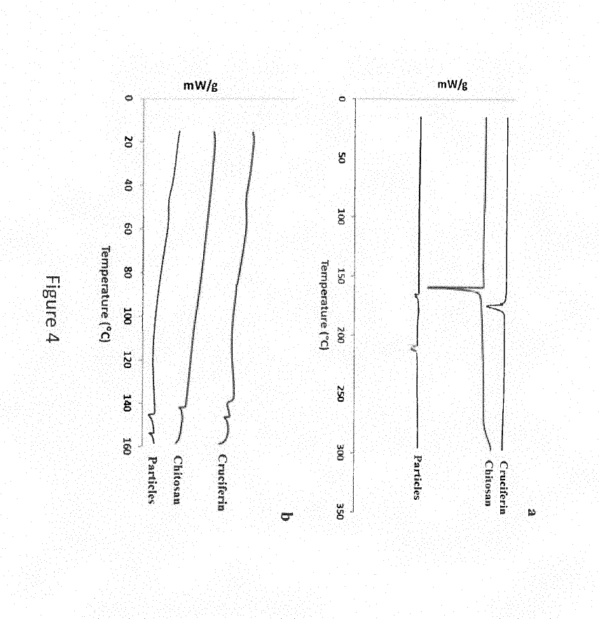

The denaturation temperature of cruciferin powder was recorded at 174° C. (FIG. 4) whereas it was previously reported 91° C. at 10% cruciferin solution. The presence of the moisture might be the reason for the difference observed in the thermal properties. Similar results were reported for dry sunflower protein isolate (181° C.), lysozyme (200° C.), ovalbumin (208° C.) and bovine serum albumin (195° C.) whereas the denaturation of these proteins occurred below 100° C. in aqueous solution. The midpoint of protein denaturation peaks is often called protein's melting temperature, drawing an analogy with melting points in solids. The melting points of chitosan and the particles were also observed at 159 and 209° C., respectively (FIG. 4a). The remarkable red shift of the melting point of the particles compared to cruciferin and chitosan suggested the formation of a new structure with enhanced thermal stability; the stability might be due to strong interactions between cruciferin and chitosan. Improvement in the thermal stability was also previously shown for the complexes of soy protein and chitosan and carboxy methylcellulose and β-lactoglobulin. The presence of a small peak at 166° C. in the particles thermogram could be attributed to the free form of cruciferin and/or chitosan which didn't contribute in the cruciferin-chitosan interactions. In the range of 15-160° C. (prior to the melting points), the DSC thermograms showed endothermic peaks corresponding to bound water evaporation temperatures from the polymer molecules (FIG. 4b). While bound water was evaporated in the range of 139-142° C. from cruciferin and chitosan powders, the evaporation peak was recorded at 146° C. for the particles. Similar results were also reported for catechin-loaded chitosan particles. The increased water evaporation temperature in the particles' thermograms might be due to the presence of more hydrophilic groups which strengthened the affinity of water binding to the particles.

### Example 9—FTIR Study

Freeze-dried samples were dried in the vacuum desiccator before FTIR analysis. The milled samples in KBr (˜10 mg) were loaded on a Thermo Nicolet 8700 FTIR spectrometer (Madison, Wis., USA). A background spectrum in air was obtained before running the samples. The infrared spectra were collected from the wavenumber of 400 to 4000 cm⁻¹ at a resolution of 4 cm⁻¹. Each sample was subjected to 64 repeated scans. The spectra were smoothed and standardized, and their baselines were calibrated using Omnic 8.1 software (Madison, Wis., USA). The recorded spectra were also deconvoluted in amide I band region (1700-1600 cm⁻¹) using the software at a bandwidth of 25 cm⁻¹ and an enhancement factor of 2.5. The detected amide I bands in the spectra were assigned to protein secondary structures using previously established wavenumber ranges.

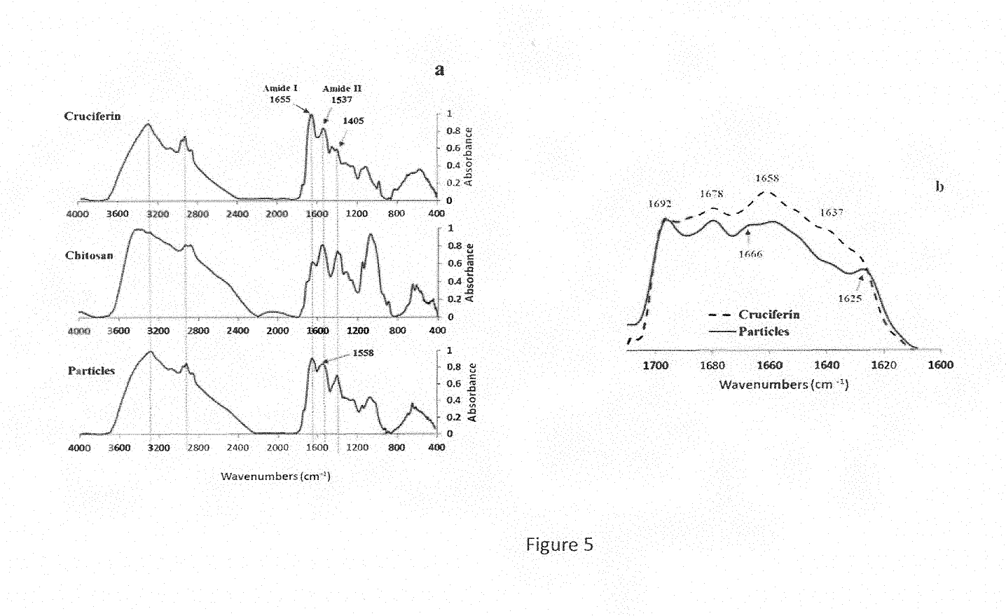

In FTIR spectra, amide I (1600-1700 cm⁻¹) and amide II (1480-1545 cm⁻¹), corresponding respectively to C═O stretching and N—H bending, provide useful information about structural changes in polymers. In comparison with amide I, amide II has much less sensitivity to conformational changes but it is more sensitive to hydrogen-bonding changes in the environment of N—H groups. In our study, while amide I band in the spectra of cruciferin, chitosan and the particles shifted in a small range of 1654-1656 cm⁻¹, amide II peak shifted from 1537 cm⁻¹ wavenumber in cruciferin spectrum to 1558 cm⁻¹ in the particles (FIG. 5a). The shift of amide II peak indicated enhanced hydrogen bonding in the formation of cruciferin-coated chitosan particles, which was in agreement with our driving forces study showing that hydrogen bonding is the main driving force involved in the particles formation. However, the very small shift observed between the amide I bands of cruciferin and the particles revealed that the conformation of cruciferin didn't significantly change in particles formation. The result is also supported by our surface hydrophobicity and intrinsic fluorescence studies. The intensity of the peak at 1405 cm⁻¹, which is a joint contribution of vibration of —OH and —CH, increased in the particles spectrum compared to that in cruciferin spectrum. The increased intensity might also be due to formation of hydrogen bonding interactions between cruciferin and chitosan. To study the small conformational changes, the secondary structure of cruciferin powder and the particles were also evaluated using deconvoluted FTIR spectra of amide I band, and were compared with the previously established wavenumber ranges. The strong absorption at 1658 cm⁻¹ indicated that α-helix was predominant in cruciferin, although the bands of 1637, 1692 and 1678 cm⁻¹ representing β-sheets and turns were also observed (FIG. 5b). In complex with chitosan, the intensity of the β-turn peak (1678 cm⁻¹) increased and new peaks were appeared at 1625 and 1666 cm⁻¹ assigning to the new structures of β-sheet and turns, respectively which are mainly formed through hydrogen bonding. The appearance of the new peaks indicated an ordered structure formed in the particles due mainly to enhanced hydrogen bonding.

### Example 10—Effect of the Particles on Cell Viability

Toxicity of the prepared particles on Caco-2 cells was assessed using MTT method developed by Mosmann (1983) with some modifications.²³ The MTT test measures the activity of dehydrogenase enzyme, which can convert MTT to insoluble purple formazan in Caco-2 cells. The formazan is dissolved in DMSO and the intensity of formed purple color determines the enzyme activity and cell viability. Caco-2 cells were seeded (50,000 cell/mL) in high-glucose and L-glutamine DMEM medium supplemented with 10% FBS, 1% non-essential amino acids, 1% penicillin and streptomycin, and 2.5% HEPS in a 96-well plate (200 μl/cell) and incubated at 37° C. in a humidified incubator with 5% CO₂ for 24 h. After incubation, the media in the cells was replaced with freshly prepared particles suspended in the DMEM medium at different concentrations of 0.5, 1.5, and 2.5 mg/mL. DMEM without nanoparticles was used as control. After 24 h incubation, the wells were gently washed three times with PBS, and then 15 μl MTT (5 mg/mL in PBS) and 185 μl medium were added to the cells and incubated for 4 h. Medium was removed and the formed formazan crystals were dissolved in 150 μl DMSO. Absorbance was measured at 570 nm using a microplate reader (GENios, Tecan, Mannedorf, Switzerland). Relative cell viability (%) was calculated by comparing the absorbance of the particle samples with that of the control.

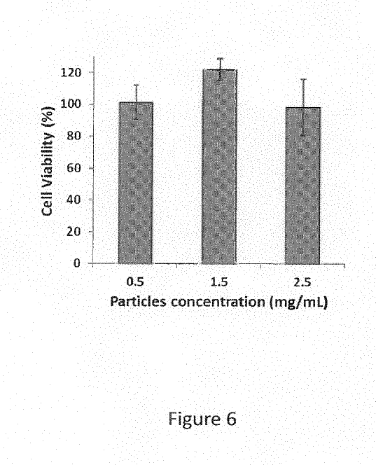

Incubation of the particles at three concentrations of 0.5, 1.5, and 2.5 mg/mL for 24 h didn't show any significant change in the cellular viability compared to the control (FIG. 6), suggesting that the particles were not toxic to the Caco-2 cells.

### Example 11—Cell Uptake of the Particles

To study the cell uptake of nanoparticles in Caco-2 cells, coumarin-6, as a fluorescent marker, was encapsulated into the nanoparticles. The cellular uptake of coumarin-6-loaded nanoparticles was compared with free coumarin-6 (not encapsulated). In brief, 1 mL of coumarin-6 solution in ethanol at concentrations ranging from 10 to 400 μg/mL was mixed with 9 mL 2 mg/mL chitosan solution in 0.1 M acetic acid. 9 mL of 5 mg/mL cruciferin solution (solubilized at pH 12) was added dropwise to the solution while stirring. The pH of the suspension was adjusted to 5.5, stirred for 2 h and then centrifuged at 40000 g and 4° C. for 30 min, Loaded particles pellet was collected, washed twice with water and centrifuged to remove free coumarin-6. The labelled particles were re-suspended in DMEM medium at concentrations of 0.1, 0.5 and 1 mg/mL. The size and zeta potential of the re-suspended particles were measured. Caco-2 cells (10⁵ cells/well) were seeded on cover glasses placed in 6-well plates (Costar, Corning, N.Y.), and incubated until cells were about 80% confluent. 2.5 mL of labelled-particles suspensions or free coumarin-6 solution in DMEM medium was added to each well and incubated for 6 h at 37° C. Then, the cells were washed three times with PBS to remove free nanoparticles and coumarin-6. Cell membranes were stained with 10 μg/mL Alexa Fluor 594-Concanavalin A conjugate in PBS for 10 min. After three washings with PBS, the cells were fixed using ice-cold solution of 3.7% formaldehyde for 30 min, Cell nuclei were also stained with 0.3 μg/mL DAPI for 10 min and three washings with PBS. The coverslips covered with fixed cells were removed from the wells, inverted on slides containing 40 μL (90% glycerol in PBS), then dried overnight in dark at room temperature, and kept at 4° C. The cells were observed with a CLSM 510 Meta confocal laser scanning microscope (Carl Zeiss Microscopy, Jena, Germany) using an oil immersion objective (63×) at wavelengths of 405, 561 and 488 nm to visualize cell nuclei, membranes and labelled particles, respectively. Images were processed with ZEN 2011 LE software (Carl Zeiss, AG, Oberkochen, Germany).

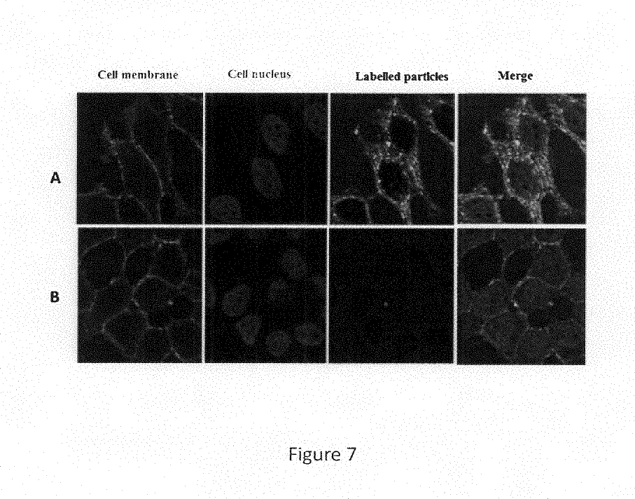

Confocal microscopy images of Caco-2 cells after 6 h incubation with coumarin-6-loaded particles and free coumarin-6 were shown in FIGS. 7A and 7B. The final concentration of the particles and coumarin-6 in the incubating DMEM medium were 0.5 mg/mL and 2.5 μg/mL, respectively. The size and zeta potential of the re-suspended particles are 304±41 nm and +7.9±2.2 mV, respectively. Since the pH of the suspension was close to cruciferin isoelectric point (pH 7.2), the zeta potential of the particles decreased, and probably due to the decrease of repulsive forces, the size of particles increased. While remarkable fluorescence intensity was observed within the cells treated with the loaded particles, no noticeable signal was detected in the cells incubated with free coumarin-6. The results showed that the particles were uptaken by cells and mostly delivered into the cells cytoplasm. This implied that the particles might be an appropriate carrier for delivery of encapsulated compounds to the cells.

### Example 12—Encapsulation of a Water-Soluble Model Compound

Brilliant blue-loaded particles were prepared by adding dropwise 18 mL of cruciferin solution (pH 12) to 18 mL chitosan solution (2 mg/mL in 0.1 M acetic acid) containing brilliant blue (1 mg/mL) and 0.04% (w/v) sodium azide, and then the pH was adjusted to 5.5. After stirring for 30 min, the particle suspension was centrifuged at 40000 g, 4° C. for 30 min, and the particle pellet was collected, washed twice with water (the same pH as suspension), and centrifuged to remove the adsorbed brilliant blue on the particle surface. The content of free brilliant blue in the combined supernatants was measured using the spectrophotometer at 590 nm. Encapsulation efficiency (EE), loading capacity (LC), and particle preparation yield (PPY) were calculated using the following equations:

EE (%)=100×(A−B)/A

LC (%)=100×(A−B)/C

PPY (%)=100×C/D
Where, A is mg model compound added to the initial suspension, B is mg free compound (unencapsulated), C is the weight (mg) of dried loaded particles, and D is the total initial dry weight (mg) of all the compounds forming the particles.

### Example 13—Encapsulation of a Water-Insoluble Model Compound

Encapsulating β-carotene in particles was performed by slowly adding 2 mL β-carotene ethanol solution (0.5 mg/mL) to 18 mL chitosan (2 mg/mL in 0.1 M acetic acid), and then 18 mL of cruciferin solution (solubilized at pH 12) was added dropwise, the pH of the suspension was adjusted to 5.5, stirred for 30 min, and then concentrated to 30 mL using a rotary vacuum evaporator (Heidolph 2-Collegiate, Germany) at 50° C. 1 mL of the concentrated suspension containing the loaded particles was mixed with 5 mL hexane, vortexed for 10 sec and then centrifuged at 10,000 rpm for 20 min at 4° C. The β-carotene extracted in the organic phase represented both unencapsulated and loosely-adsorbed β-carotene on the surface of particles. Absorbance of the organic phases was measured at 450 nm using the UV-VIS spectrometer and EE, LC and PPY were calculated.

### Example 14—Simulated Gastro-Intestinal Release Study

Release experiments were performed in simulated gastric fluid (SGF) followed by simulated intestinal fluid (SIF). Brilliant blue- and β-carotene-loaded particles were prepared using the previously described method. To simulate gastric condition, 20 mL of above suspensions was adjusted to pH 1.4 using 10 mL of 0.05 M HCl containing 1 mg/mL pepsin and was stirred at 100 rpm and 37° C. in a water bath. Release studies were also performed at pH 1.4 and in the absence of pepsin. 0.5 mL samples were withdrawn from the release media at predetermined intervals. 0.25 mL of 1 M sodium bicarbonate was added to the samples to increase pH to 7.4 and inactivate pepsin. Brilliant blue-containing samples were centrifuged (40000 g) and released brilliant blue in supernatant was measured. β-carotene was extracted from the withdrawn samples using 2.5 mL hexane and quantified using the method previously described. After 2 h incubation in SGF, pepsin was inactivated by raising pH to 7.4 using 10 mL of 1 M sodium bicarbonate and release studies were followed in SIF medium by adding 5 ml of 200 mM phosphate buffer pH 7.4 in the absence and presence of 2 mg/mL pancreatin. 0.5 mL samples were withdrawn at predetermined intervals. To stop pancreatin activity after sampling, brilliant blue-containing samples were heated at 90° C. for 5 min while for β-carotene samples, free β-carotene was immediately extracted using hexane. The concentrations of released brilliant blue and β-carotene in SGF and following SIF media were measured and their cumulative percentages were calculated based on the total amount of the initial loaded compounds and the released compounds in recovery studies.

Encapsulation property and release behaviour, two important factors affecting the efficiency of a delivery system, were studied for the particles. Brilliant blue and β-carotene, representing hydrophilic and hydrophobic model compounds, were encapsulated in the particles. While the encapsulation efficiency (EE), loading capacity (LC) and particle preparation yield (PPY) of brilliant blue were 72.4%±0.7, 16.3%±0.8 and 55.5%±2.6, those for β-carotene were 98.8%±0.2, 7.7%±0.2 and 44.5%±1.1, respectively. Previously the EE of β-carotene was reported to be 39.5%±0.3 in chitosan-β-lactoglobulin nanoparticles, 54.7% and 87.6%, respectively, in chitosan-alginate beads and sodium caseinate sub-micelles. The higher EE of the particles suggested that the particles are appropriate carriers for both water-soluble and -insoluble compounds.

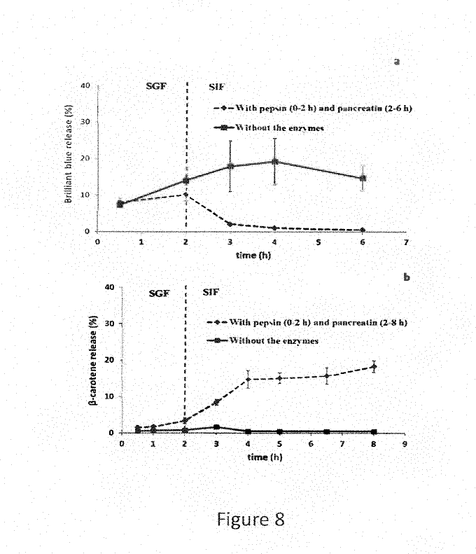

In vitro release of brilliant blue and β-carotene from the particles was studied using 2 h incubation in SGF followed by 6 h in SIF with and without pepsin and pancreatin, respectively (FIG. 8). In SGF (in the presence or absence of pepsin), less than 10% and 5% of encapsulated brilliant blue and β-carotene were released in the first 2 h, due to the weakly attached compounds on the surface of the particles. These results suggested that the particles were resistant to both low pH and pepsin. Since chitosan is degraded at low pH³, the particles resistance in the SGF further supported that chitosan was coated by cruciferin layers. The coating layer might be a cruciferin layer but not a complex of cruciferin and chitosan since the complex can be degraded at pH 1.2 due to high protonation of chitosan (pKa˜6.4) and cruciferin (IEP ˜7.2) and increased repulsive forces. This result further bolstered our previous surface hydrophobicity results showing the presence of a cruciferin layer on the particles surface. The indigestibility of cruciferin in the stomach was reported in our previous study and also by Bos et al. (2007). Our results suggested that cruciferin could be applied as a protective polymer for coating chitosan particles and its protection effect is comparable with other coating polymers such as hydroxypropyl methylcellulose phthalate (a pH-sensitive polymer); where in a previous study, 25% of encapsulated insulin was released from chitosan/hydroxypropyl methylcellulose phthalate particles.

In SIF (pH 7.4) and in the absence of pancreatin, the particles stayed resistant and only less than 5-10% of encapsulated brilliant blue and B-carotene were released (FIG. 8).

However, in the presence of pancreatin, the previously released brilliant blue (during in SGF) was re-adsorbed by the particles and precipitated along with particles after centrifugation. The reason might be due to the degradation of the cruciferin layer by pancreatin which released positively-charged amine groups of chitosan and then bound to the previously released negatively-charged brilliant blue. Similar results were reported by Chen and Subirade (2005).

However, unlike brilliant blue-loaded particles, degradation of the cruciferin layer by pancreatin released ˜15% of β-carotene in SIF. The released β-carotene from enzymatic-hydrolyzed particles might be related to the β-carotene which was entrapped in the cruciferin coating layer. Afterward, the remaining encapsulated β-carotene, entrapped in the chitosan-based core, was not released since chitosan was resistant to the pH and enzymes in SIP. Similar results were reported in a chitosan dispersed system coated with aminoalkyl methacrylate copolymer RS (Eudragit RS) and lactose-sodium alginate-chitosan composite particles. Therefore, the particles are promising carriers for delivery of sensitive compounds to both gastric and intestinal conditions such as protein-based bioactive compounds.

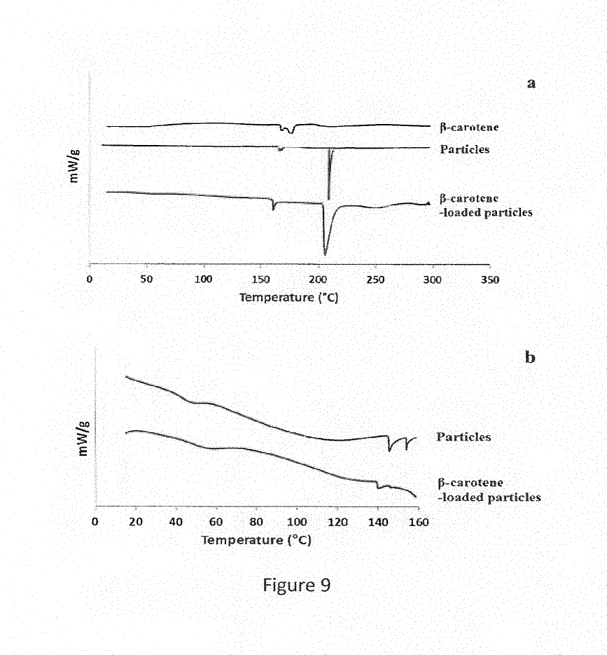

The encapsulated β-carotene might affect the thermal property and structure of the particles. In the study of the DSC thermograms, pure crystalline β-carotene showed two melting peaks at 168 and 176° C. (FIG. 9a). After dissolving β-carotene in ethanol and encapsulation in the particles, its melting point decreased to 160° C. The melting point of loaded particles was 205° C. compared to 209° C. of empty particles indicating less crystalline structure in the loaded particles. Transition of β-carotene molecules from crystalline powder to its amorphous form during encapsulation process might be the reason for the decreased melting point. Similar melting points of 178-180° C. and 150-160° C. were also reported by Coronel-Aguilera and Martin Gonzalez (2015) and de Paz et al. (2012) for pure (crystalline) and encapsulated (amorphous) forms of β-carotene, respectively.

At the temperature range of 15-160° C. (prior to the melting points), endothermic water evaporation peaks were presented at 146° C. and 140° C. for empty and β-carotene-loaded particles, respectively (FIG. 9b). The decrease in the water evaporation temperature might be due to the presence of β-carotene aliphatic chain and its hydrophobic groups which had less affinity to water and decreased the evaporation temperature. Similar observations were also reported by Luo et al. (2011) and Paula et al. (2011) in encapsulation of vitamin D3 and essential oils, respectively.

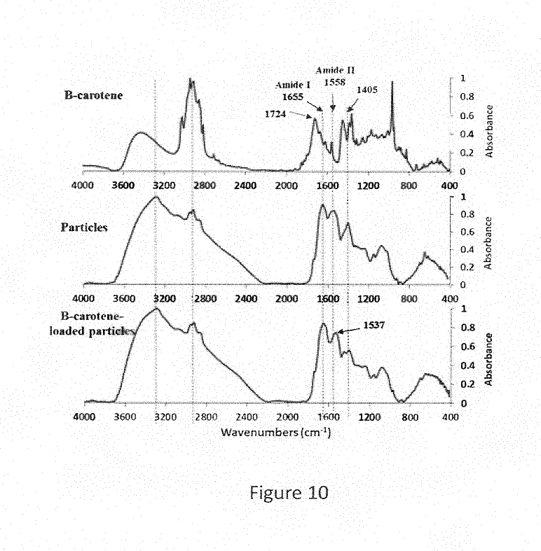

To study the effect of the encapsulation on the structure of the particles, the FTIR spectra of β-carotene, particles and β-carotene-loaded particles were compared in FIG. 10. Two strong peaks were observed at 965 and 2925 cm⁻¹ in the β-carotene spectrum representing trans conjugated alkene CH and CH stretching vibration, respectively. The disappearance of 965 and 1724 cm⁻¹ peaks in the β-carotene-loaded particles spectrum indicated successful encapsulation of β-carotene in the particles. Similar result was shown by Teng et al., (2013) in encapsulation of D₃ in chitosan/soy protein complex. Evaluation of the spectrum of β-carotene-loaded particles also showed that no shift was observed in the amide I peak compared to that of particles, indicating that the incorporation of β-carotene didn't affect the conformation of the protein. However, a redshift of amide II band of loaded-particles compared to that of empty particles (from 1558 to 1537 cm⁻¹) revealed that the environment of N—H groups changed favouring weaker hydrogen bonding. The intensity of 1405 cm⁻¹ peak was also decreased after β-carotene incorporation, further supporting decreased hydrogen bonding. The decreased hydrogen bonding in the loaded particles might be due to the presence of long aliphatic chain of β-carotene which weakened the initial hydrogen interactions present in the particles. Luo et al. (2011) also reported that amide I and II in the spectrum of α-tocopherol-loaded zein particles redshifted to lower frequencies compared to those in zein spectrum. They suggested that electrostatic interaction is involved in the α-tocopherol encapsulation. However, Yi et al. (2015) showed that the incorporation of β-carotene in casein, whey and soy protein particles shifted amide I and amide II respectively to lower and higher frequencies compared to empty particles.

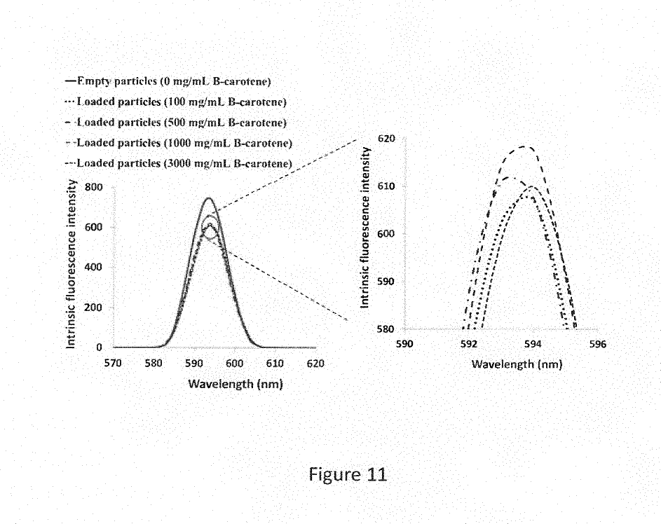

Binding of β-carotene to the particles can also be characterized by determination of intrinsic fluorescence of tryptophan residues in cruciferin structure. The intensity of particles was initially decreased after encapsulation of β-carotene (FIG. 11). However, unlike the results reported by Perez et al. (2014) and Zimet and Livney (2009) showing that the fluorescence intensity of loaded particles decreased does-dependently at increasing loaded compounds concentrations due to the quenching effect, the emission intensity of β-carotene-loaded particles didn't change at increasing concentrations of loaded β-carotene. The initial decrease observed in the intensity after adding β-carotene suggested that a small part of β-carotene was bound to cruciferin coating and led to quenching effect; while, most of loaded β-carotene didn't interact with cruciferin and the binding occurred in chitosan core part and in other words, β-carotene was loaded in chitosan-based core of the particles. This confirms that the 15% released β-carotene in SIF (with enzyme) was released from cruciferin coating layer and the remaining 80-85% β-carotene was entrapped in chitosan core which was not affected by the enzyme and/or pH 7.4.

### Example 15—Statistical Analysis

All Experiments were carried out in triplicates and results were statistically analyzed using ANOVA and Duncan tests (version 9.2, SAS Institute Inc., Cary, N.C., USA).

### Definitions and Interpretation

The description of the present invention has been presented for purposes of illustration and description, but it is not intended to be exhaustive or limited to the invention in the form disclosed. Many modifications and variations will be apparent to those of ordinary skill in the art without departing from the scope and spirit of the invention. Embodiments were chosen and described in order to best explain the principles of the invention and the practical application, and to enable others of ordinary skill in the art to understand the invention for various embodiments with various modifications as are suited to the particular use contemplated.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such aspect, feature, structure, or characteristic with other embodiments, whether or not explicitly described. In other words, any element or feature may be combined with any other element or feature in different embodiments, unless there is an obvious or inherent incompatibility between the two, or it is specifically excluded.

It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition or step being referred to is an optional (not required) feature of the invention.

The singular forms “a,” “an,” and “the” include the plural reference unless the context clearly dictates otherwise. The term “and/or” means any one of the items, any combination of the items, or all of the items with which this term is associated. The phrase “one or more” is readily understood by one of skill in the art, particularly when read in context of its usage.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

### REFERENCES

The following references are incorporated herein by reference (where permitted) as if reproduced in their entirety. All references are indicative of the level of skill of those skilled in the art to which this invention pertains.
1 H. Bouwmeester, S. Dekkers, M. Y. Noordam, W. I. Hagens, A. S. Bulder, C. de Heer, S. E. C. G. ten Voorde, S. W. P. Wijnhoven, H. J. P. Marvin and A, J. A. M. Sips, Regulatory Toxicology and Pharmacology, 2009, 53, 52-62.
2 Z. Teng, Y. Luo and Q. Wang, Food Chem., 2013, 141, 524-532.
3 A. Makhlof, Y. Tozuka and H. Takeuchi, European Journal of Pharmaceutical Sciences, 2011, 42, 445-451.
4 M. K. Chourasia and S. K. Jain, Journal of Pharmacy and Pharmaceutical Sciences, 2003, 6, 33-66,
5 A. O. Elzoghby, W. M. Samy and N. A. Elgindy, J. Controlled Release, 2012, 161, 38-49,
6 USDA. United States Department of Agriculture, http://apps.fas.usda.gov/psdonline/circulars/oilseeds.pdf, (accessed December, 2013).
7 J. Wu and A. D. Muir, J. Food Sci., 2008, 73, C210-C216.
8 S. H. Tan, R. J. Mailer, C. L. Blanchard and S. O. Agboola, Food Res, Int., 2011, 44, 1075-1082.
9 J. Wu, R. E. Aluko and A. D. Muir, Bioresour, Technol., 2009, 100, 5283-5287,
10 Y. Yamada, M. Iwasaki, H. Usui, K. Ohinata, E. D, Marczak, A. W. Lipkowski and M. Yoshikawa, Peptides, 2010, 31, 909-914.
11 Y. Yoshie-Stark, Y, Wada and A. Waesche, Food Chem., 2008, 107, 32-39.
12 A. Pinterits and S. D. Arntfield, Food Chem., 2007, 102, 1337-1343.
13 C. Bos, G. Airinei, F. Mariotti, R. Benamouzig, S. Berot, J. Evrard, E. Fenart, D. Tome and C. Gaudichon, J. Nutr., 2007, 137, 594-600.
14 A. O. Elzoghby, W. S. A. El-Fotoh and N. A. Elgindy, J. Controlled Release, 2011, 153, 206-216.
15 A. Akbari and J. Wu, Lwt-Food Science and Technology, 2015, 64, 308-315.
16 P. S. Lee, S. G. Yim, Y. Choi, Thi Van Anh Ha and S. Ko, Food Chem., 2012, 134, 992-998.
17 N. Alizadeh-Pasdar and E. C. Y. Li-Chan, J. Agric, Food Chem., 2000, 48, 328-334.
18 A. A. Perez, R. B. Andermatten, A. C. Rubiolo and L. G. Santiago, Food Chem., 2014, 158, 66-72.
19 P. Zimet and Y. D. Livney, Food Hydrocoll., 2009, 23, 1120-1126.
20 C. Schmitt, C, Moitzi, C. Bovay, M. Rouvet, L. Bovetto, L. Donato, M. E. Leser, P. Schurtenberger and A. Stradner, Soft Matter, 2010, 6, 4876-4884.
21 J. T. Pelton and L. R. McLean, Anal. Biochem., 2000, 277, 167-176,
22 J. Kong and S. Yu, Acta Biochimica Et Biophysica Sinica, 2007, 39, 549-559.
23 T. Mosmann, J. Immunol Methods, 1983, 65, 55-63.
24 Y. Luo, Z. Teng, T. T. Y. Wang and Q. Wang, J Agric. Food Chem., 2013, 61, 7621-7629.
25 B. He, P. Lin, Z. Jia, W. Du, W. Qu, L. Yuan, W. Dai, H. Zhang, X. Wang, J. Wang, X. Zhang and Q. Zhang, Biomaterials, 2013, 34, 6082-6098.
26 M. Gaumet, R. Gurny and F. Delie, European Journal of Pharmaceutical Sciences, 2009, 36, 465-473.
27 A, Mensi, Y. Choiset, T. Haertle, E. Reboul, P. Borel, C. Guyon, M. de Lamballerie and J. Chobert, Food Chem., 2013, 139, 253-260.
28 L. Y. Chen and M. Subirade, Biomaterials, 2005, 26, 6041-6053.
29 V. Boeris, Y. Micheletto, M. Lionzo, N. P. da Silveira and G. Pico, Carbohydr. Polym., 2011, 84, 459-464.
30 S. Gorinstein, I, Goshev, S. Moncheva, M. Zemser, M. Weisz, A. Caspi, I. Libman, H, T. Lerner, S. Trakhtenberg and O. Martin-Belloso, J. Protein Chem., 2000, 19, 637-642.
31 M, van de Weert and L. Stella, J. Mol. Struct., 2011, 998, 144-150.
32 Y. Qin, L. Chen, X. Wang, X. Zhao and F. Wang, Carbohydr, Polym., 2011, 84, 329-334.
33 Y. M. Xu and Y. M. Du, Int. J. Pharm., 2003, 250, 215-226.
34 J. M. Bier, C. J. R. Verbeek and M. C. Lay, Macromolecular Materials and Engineering, 2014, 299, 524-539,
35 A. Rouilly, O. Orliac, F. Silvestre and L. Rigal, Polymer, 2001, 42, 10111-10117.
36 J. Carpenter, D. Katayama, L. Liu, W. Chonkaew and K. Menard, Journal of Thermal Analysis and calorimetry, 2009, 95, 881-884.
37 G. Huang, Y. Sun, J, Xiao and J. Yang, Food Chem., 2012, 135, 534-539.
38 C. Capitani, O, E. Perez, B. Pacheco, M. Teresa and A. M. R. Pilosof, Food Hydrocoll., 2007, 21, 1344-1354.
39 A. R. Dudhani and S. L. Kosaraju, Carbohydr, Polym., 2010, 81, 243-251.
40 Y, Luo, B. Zhang, M, Whent, L, Yu and Q. Wang, Colloids and Surfaces B-Biointerfaces, 2011, 85, 145-152,
41 A. Almutawah, S. A. Barker and P. S. Belton, Biomacromolecules, 2007, 8, 1601-1606.
42 A. Ullah, T. Vasanthan, D. Bressler, A, L. Elias and J, Wu, Biomacromolecules, 2011, 12, 3826-3832.
43 V. Gilbert, M, Rouabhia, H, X. Wang, A. L, Arnould, G, Remondetto and M. Subirade, Biomaterials, 2005, 26, 7471-7480.
44 B. Chu, S. Ichikawa, S. Kanafusa and M. Nakajima, J. Agric. Food Chem., 2007, 55, 6754-6760.
45 E. G. Donhowe, F. P. Flores, W. L. Kerr, L. Wicker and F. Kong, Lwt-Food Science and Technology, 2014, 57, 42-48.
46 A. Akbari and J. Wu, Food Hydrocoll., 2016, 54, 107-118.
47 N. Shimono, T. Takatori, M. Ueda, M. Mori, Y. Higashi and Y. Nakamura, Int. J. Pharm., 2002, 245, 45-54.
48 H. Takeuchi, J. Thongborisute, Y. Matsui, H. Sugihara, H. Yamamoto and Y, Kawashima, Adv. Drug Deliv. Rev., 2005, 57, 1583-1594.
49 C. P. Coronel-Aguilera and M, F. S. Martin-Gonzalez, Lwt-Food Science and Technology, 2015, 62, 187-193.
50 E. de Paz, A. Martin, A. Estrella, S. Rodriguez-Rojo, A. A. Matias, C. M. M. Duarte and M. Jose Cocero, Food Hydrocoll., 2012, 26, 17-27.
51 H. C. B. Paula, F. M. Sombra, R. d. F, Cavalcante, F. O. M. S. Abreu and R. C. M. de Paula, Materials Science & Engineering C-Materials for Biological Applications, 2011, 31, 173-178.
52 J. Yi, T. I, Lam, W. Yokoyama, L. W, Cheng and F. Zhong, Food Hydrocoll., 2015, 43, 31-40.
53 S. Schlucker, A. Szeghalmi, M. Schmitt, J. Popp and W. Kiefer, J. Raman Spectrosc., 2003, 34, 413-419.
54 M. Keeney, X. Y. Jiang, M. Yamane, M., Lee, S. Goodman and F. Yang, J Mater. Chem. B, 2015, 3, 8757-8770.
55 E. M. Shchukina and D. G. Shchukin, Curr Opin. Colloid Interface Sci., 2012, 17, 281-289.
56 S. Abdul and S. S. Poddar, J. Controlled Release, 2004, 97, 393-405,
57 K. Y. Win and S. S. Feng, Biomaterials, 2005, 26, 2713-2722.

What is claimed is:

1. A nanoparticle comprising cruciferin and chitosan.

2. The nanoparticle of claim 1 further comprising a bioactive material.

3. The nanoparticle of claim 2 wherein the bioactive material is a compound or a probiotic, and is incorporated into the nanoparticle during formation of the nanoparticle.

4. The nanoparticle of claim 1 wherein the nanoparticle comprises a shell comprising cruciferin which wholly or partially encapsulates a core comprising chitosan.

5. The nanoparticle of claim 4 wherein the bioactive material is associated with the chitosan.

6. The nanoparticle of claim 2 which is resistant to Simulated Gastric Fluid H, adjusted to pH 1.4 using 0.05 M HCl, containing 1 mg/mL pepsin and stirred at 100 rpm and 37° C. in a water bath, such that less than 10% of the bioactive material is released after 2 hours.

7. A method of forming a nanoparticle for delivery of a bioactive material, comprising the steps of:
(a) combining a solution of cruciferin and a solution of chitosan in a weight ratio greater than 1:1 and less than about 5:1, and at a pH greater than 4.5 and less than 7.5; and
(b) collecting the resulting nanoparticles.

8. The method of claim 7 wherein the weight ratio of cruciferin to chitosan is in the range of about 3:2 to about 5:2 by weight.

9. The method of claim 8 wherein the pH of the combined solution is adjusted to between about 5.5 to about 6.5.

10. The method of claim 7 wherein the cruciferin solution is sufficiently alkaline to be negatively-charged and the chitosan solution is sufficiently acidic to be positively-charged, and the resulting mixture is at a pH where cruciferin remains negatively charged and chitosan remains positively charged.
